(19) United States
(12) Patent Application Publication
(10) Pub. No.: US 2025/0319038 A1
(43) Pub. Date: Oct. 16, 2025

(54) METHOD FOR PREPARING EXTRACELLULAR MATRIX NANOPARTICLE AND METHODS OF PREVENTING OR TREATING LUNG DISEASE AND REPAIRING HAIR FOLLICLE

(71) Applicant: National Taiwan University of Science and Technology, TAIPEI CITY (TW)

(72) Inventors: Chen-Yu KAO, Taipei City (TW); Hsiu-Yi CHIN, Taoyuan City (TW)

(21) Appl. No.: 18/827,872

(22) Filed: Sep. 9, 2024

(30) Foreign Application Priority Data
Apr. 16, 2024 (TW) 113114115

(51) Int. Cl. A61K 9/51; A61K 9/00; A61K 35/22; A61K 35/42; A61P 11/00

(52) U.S. Cl. CPC A61K 9/5176; A61K 9/0053; A61K 9/5192; A61K 35/22; A61K 35/42; A61P 11/00

(57) ABSTRACT

Embodiments of this disclosure provide a method for preparing extracellular matrix nanoparticles, including the following steps. Visceral tissues of a pig are provided. The visceral tissues are homogenized. The visceral tissues are decellularized to obtain an extracellular matrix. The extracellular matrix is lyophilized, and the extracellular matrix lyophilized is ground into powder. A digestion process is performed on the extracellular matrix ground through an acidic solution to produce an acidic extracellular matrix. A neutralization process is performed on the acidic extracellular matrix through an alkaline solution to produce an extracellular matrix hydrogel. The extracellular matrix hydrogel is cultured to gelatinize the extracellular matrix hydrogel. A sonication process is performed on the extracellular matrix hydrogel to make the extracellular matrix hydrogel into a plurality of extracellular matrix nanoparticles.

### CROSS-REFERENCE TO RELATED APPLICATION

This application claims priority to Taiwan Application No. 113114115, filed Apr. 16, 2024, which is herein incorporated by reference in its entirety.

### BACKGROUND

### Field of Invention

The present disclosure relates to a method for preparing extracellular matrix nanoparticle and methods of preventing or treating lung disease and repairing hair follicle.

### Description of Related Art

At present, according to literatures and liquid chromatography mass spectrometry (LCMS) analysis, it can be seen that collagen compositions of porcine bladder tissues and porcine lung tissues are different. The porcine bladder tissues mostly include type-IV collagen. The porcine lung tissues mostly include type-I collagen and elastin. Therefore, the porcine bladder tissues and the porcine lung tissues are used in different ways. For example, type-I collagen is accounted for 90% of a total amount of human body. Type-I collagen mainly acts on skin, teeth, hair, ligaments, blood vessels, etc., to help heal wounds when injured, and type-I collagen has the most uses for collagen.

However, the porcine bladder tissues and the porcine lung tissues are rarely used for food and are usually discarded. To avoid waste and increase the economic value of the porcine bladder tissues and the porcine lung tissues, this disclosure provides a method for preparing extracellular matrix nanoparticles through the porcine bladder tissues and the porcine lung tissues.

### SUMMARY

Embodiments of this disclosure provide a method for preparing extracellular matrix nanoparticles, including the following steps. Visceral tissues of a pig are provided. The visceral tissues are homogenized. The visceral tissues are decellularized to obtain an extracellular matrix. The extracellular matrix is lyophilized, and the lyophilized extracellular matrix is ground into powder. A digestion process is performed on the powdered extracellular matrix through an acidic solution and producing an acidic extracellular matrix. A neutralization process is performed on the acidic extracellular matrix through an alkaline solution to produce an extracellular matrix hydrogel. The extracellular matrix hydrogel is cultured to gelatinize the extracellular matrix hydrogel. A sonication process is performed on the extracellular matrix hydrogel to make the extracellular matrix hydrogel into a plurality of extracellular matrix nanoparticles.

Embodiments of this disclosure provide a method of preventing or treating a lung disease, and the method includes administering to a subject in need thereof an effective amount of a plurality of extracellular matrix nanoparticles. A diameter of each of the plurality of extracellular matrix nanoparticles is less than 5 micrometers.

Embodiments of this disclosure provide a method of repairing hair follicles, and the method includes administering to a subject in need thereof an effective amount of a plurality of extracellular matrix nanoparticles.

### BRIEF DESCRIPTION OF THE DRAWINGS

In order to make the above and other purposes, features, advantages and embodiments of this disclosure more clearly understandable, the accompanying figures are described as follows.

FIG. 1 is a flow chart of a method for preparing extracellular matrix nanoparticles according to some embodiments of this disclosure,

FIG. 2 is a comparative diagram of non-decellularized porcine bladder tissues and decellularized porcine bladder tissues according to some embodiments of this disclosure,

FIG. 3A is a view under a microscope of extracellular matrix nanoparticles according to some embodiments of this disclosure,

FIG. 3B is an enlarged view of extracellular matrix nanoparticles based on FIG. 3A under a microscope,

FIGS. 4A and 4B are statistical bar charts of IL-10 and TNF-α in Experiment group 1 and Control group 1 respectively obtained through qPCR,

FIGS. 4C and 4D are statistical bar charts of IL-10 and TNF-α in Experiment group 1 and Control group 1 respectively obtained through ELISA,

FIG. 5A is a bar chart related to a concentration of TNF-α in a lavage fluid of Control groups 2-1 and 2-2 and Experiment groups 2-1 to 2-4,

FIG. 5B is a bar chart related to a concentration of IL-10 in a lavage fluid of Control groups 2-1 and 2-2 and Experiment groups 2-1 to 2-4,

FIGS. 6A to 6C are section staining figures of alveolus of Experiment groups 2-1 to 2-4,

FIG. 7 is related to pictures of variations of hair follicles in a hair follicle growth experiment, and

FIG. 8 is a bar chart of hair follicle growth degrees regarding variations of hair follicles in a hair follicle growth experiment.

### DETAILED DESCRIPTION

To make the description of this disclosure more detailed and complete, the implementations and specific embodiments of this disclosure are described in detail below, but not the only form of implementing or using the specific embodiments of this disclosure. The embodiments disclosed below may be combined or replaced with each other under beneficial circumstances, and other embodiments may be added to one embodiment without further description or explanation. In the following description, multiple specific details are set forth in detail to enable readers to completely understand the following embodiments. However, embodiments of this disclosure may be practiced without these specific details.

In this specification, unless the description is specifically limited in the description, “a/an” and “the” may generally refer to one or more. It is further understood that the words “comprise”, “include”, “have” and the like used in this disclosure refer to features, areas, integer, steps, elements and/or components, and is not intended to exclude, for example, other features, areas, integer, steps, elements, components and/or a combination thereof.

Although a series of operations or steps are used to describe a method disclosed herein below, the order shown in these operations or steps shall not be construed as a limitation of this disclosure. For example, certain operations or steps may be performed in a different order and/or concurrently with other steps. Additionally, not all operations, steps, and/or features shall be performed to practice embodiments of this disclosure. Furthermore, each operation or step described herein may include several sub-steps or actions.

### I. a Method for Preparing Extracellular Matrix Nanoparticles

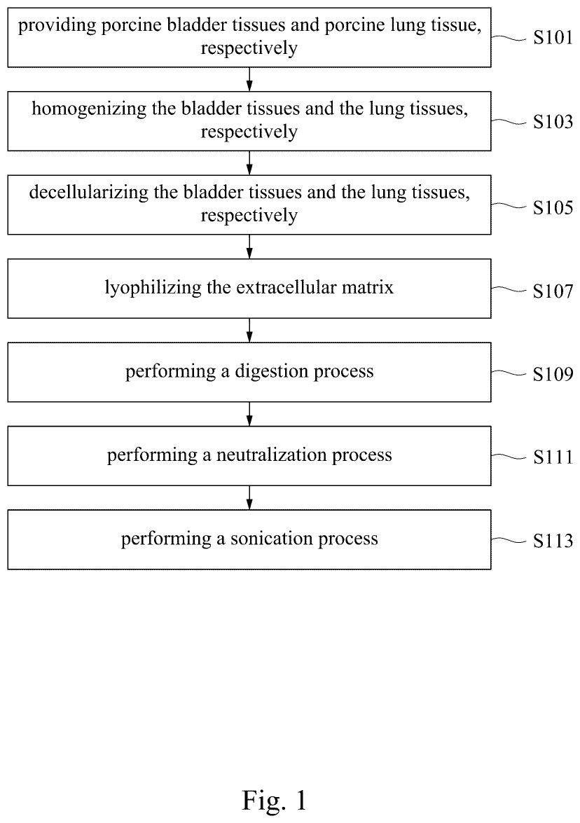

Please refer to FIG. 1. FIG. 1 is a flow chart of a method for preparing extracellular matrix nanoparticles according to some embodiments of this disclosure. Firstly, in step S101, visceral tissues of a pig were provided. In some embodiments, the visceral tissues were bladder tissues or lung tissues. In some embodiments, the bladder tissues and the lung tissues were washed with phosphate buffered saline (PBS), respectively, and the bladder tissues and the lung tissues were lyophilized, respectively. In some embodiments, a weight of the bladder tissues was 2.5 grams (g). In some embodiments, a weight of the lung tissues was 2.5 g.

In step S103, the visceral tissues were homogenized. For example, the lyophilized bladder tissues and the lyophilized lung tissues were further ground into small pieces using a laboratory grinder, respectively. In some embodiments, the lyophilized bladder tissues and the lyophilized lung tissues were ground for two minutes, respectively, a size of the bladder tissues and a size of the lung tissues were reached to from 1 micrometer (μm) to 10 μm, respectively.

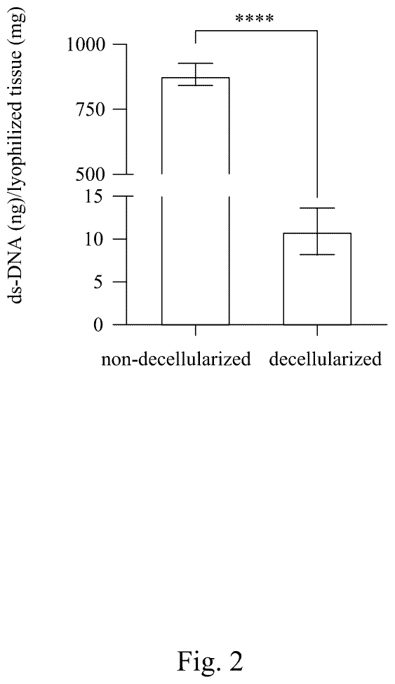

In step S105, the visceral tissues were decellularized to obtain an extracellular matrix. Specifically, the bladder tissues were decellularized to obtain a bladder extracellular matrix, and the lung tissues were decellularized to obtain a lung extracellular matrix. In some embodiments, the decellularization process included adding 1 (wt/v) % sodium dodecyl sulfate (SDS), 10% fetal calf serum (FCS), hypertonic saline solution, hypotonic saline solution, 1×PBS, deionized water (DI water) and other decellularization reagents were added. The hypertonic saline solution was referred to a saline solution with a concentration of 1.28 molarity (M), and the hypotonic saline solution was referred to a saline solution with a concentration of 0.34 M. In some embodiments, a duration time of the decellularization process was in a range from 12 hours to 24 hours, until a color of the bladder tissue and lung tissue appeared white or even transparent, respectively. Next, please refer to FIG. 2. FIG. 2 is a comparative diagram of a double-stranded DNA (dsDNA) content of non-decellularized bladder tissues and decellularized bladder tissues according to some embodiments of this disclosure. As shown in FIG. 2, more than 98% of dsDNA was removed from the bladder tissues. In addition, although FIG. 2 only shows the comparison of the bladder tissues before and after decellularization, more than 98% of dsDNA in the porcine lung tissues was also removed from the lung tissue.

In step S107, the extracellular matrix was lyophilized. Specifically, the bladder extracellular matrix and the lung extracellular matrix were lyophilized, respectively. Then, the lyophilized bladder extracellular matrix and the lyophilized lung extracellular matrix were ground into powder and stored at −20° C.

In step S109, a digestion process was performed on the extracellular matrix which was lyophilized and ground. Specifically, the powdered bladder extracellular matrix and the powdered lung extracellular matrix were performed the digestion process through an acidic solution, the digestion process was performed until the bladder extracellular matrix and the lung extracellular matrix became chylous, respectively. In other words, an acidic bladder extracellular matrix and an acidic lung extracellular matrix were formed, respectively. In some embodiments, the digestion process included performing at 37° C. for 48 hours. In some embodiments, a pH value of the acidic solution was in a range from 3 to 4. In some embodiments, the acidic solution included an acetic acid solution, a citric acid solution, a hydrochloric acid solution, a lactic acid solution, or a combination thereof. In the embodiments that the acidic solution included the hydrochloric acid solution, a concentration of the hydrochloric acid solution was 0.01 normality (N).

In step S111, a neutralization process was performed on the digested extracellular matrix. Specifically, after performing the digestion process, the neutralization process was performed on the acidic bladder extracellular matrix and the acidic lung bladder extracellular through an alkaline solution to produce a bladder extracellular matrix hydrogel and a lung extracellular matrix hydrogel, respectively. The bladder extracellular matrix hydrogel and the lung extracellular matrix hydrogel were collectively referred to as an extracellular matrix hydrogel below. In other words, the extracellular matrix hydrogel included bladder extracellular matrix hydrogel or the lung extracellular matrix hydrogel. Further, the neutralized extracellular matrix hydrogel was incubated at 37° C. for 1 hour to achieve complete gelatinization. In some embodiments, the alkaline solution included 0.01N sodium hydroxide (NaOH) and 1×PBS. In some embodiments, the neutralization process was performed at 4° C. for 1 hour. In some embodiments, after the neutralization process, a pH value of the extracellular matrix hydrogel was in a range from 7.0 to 7.4. In some embodiments, the pH of the extracellular matrix hydrogel included 7.0, 7.1, 7.2, 7.3, or 7.4. In some embodiments, preferably, the pH value of the extracellular matrix hydrogel was 7.4. In some embodiments, when a concentration of the completely gelatinized extracellular matrix hydrogel was 4 mg/mL, a storage modulus of the extracellular matrix hydrogel was in a range from 25 Pa to 35 Pa. In some embodiments, when a concentration of the completely gelatinized extracellular matrix hydrogel was 4 mg/mL, the storage modulus of the extracellular matrix hydrogel included, but was not limited to, 25.0 Pa, 25.5 Pa, 26.0 Pa, 26.5 Pa, 27.0 Pa, 27.5 Pa, 28.0 Pa, 28.5 Pa, 29.0 Pa, 29.5 Pa, 30.0 Pa, 30.5 Pa, 31.0 Pa, 31.5 Pa, 32.0 Pa, 32.5 Pa, 33.0 Pa, 33.5 Pa, 34.0 Pa, 34.5 Pa, 35.0 Pa or any value between any two foregoing storage modulus values. In some embodiments, when the concentration of the completely gelatinized extracellular matrix hydrogel was in a range from 6 mg/mL to 8 mg/mL, the storage modulus of the extracellular matrix hydrogel was in a range from 110 Pa to 120 Pa. In some embodiments, when the concentration of the completely gelatinized extracellular matrix hydrogel was in a range from 6 mg/mL to 8 mg/mL, the storage modulus of the extracellular matrix hydrogel included, but was not limited to, 110 Pa, 111 Pa, 112 Pa, 113 Pa, 114 Pa, 115 Pa, 116 Pa, 117 Pa, 118 Pa, 119 Pa, 120 Pa or any value between any two foregoing storage modulus values.

In step S113, a sonication process was performed on the extracellular matrix hydrogel to make the extracellular matrix hydrogel into a plurality of extracellular matrix nanoparticles. In some embodiments, the sonication process was performed through an ultrasonic homogenizer. In some embodiments, the sonication process was performed with a power of 25 watts (W) for 30 seconds, a dwell time of 10 seconds, and then performed with another 30 seconds, for a total of 4 cycles. In some embodiments, sonication process was performed with a power of 25 W for one minute, a dwell time of 30 seconds, and then performed with another one minute, for a total of 4 cycles.

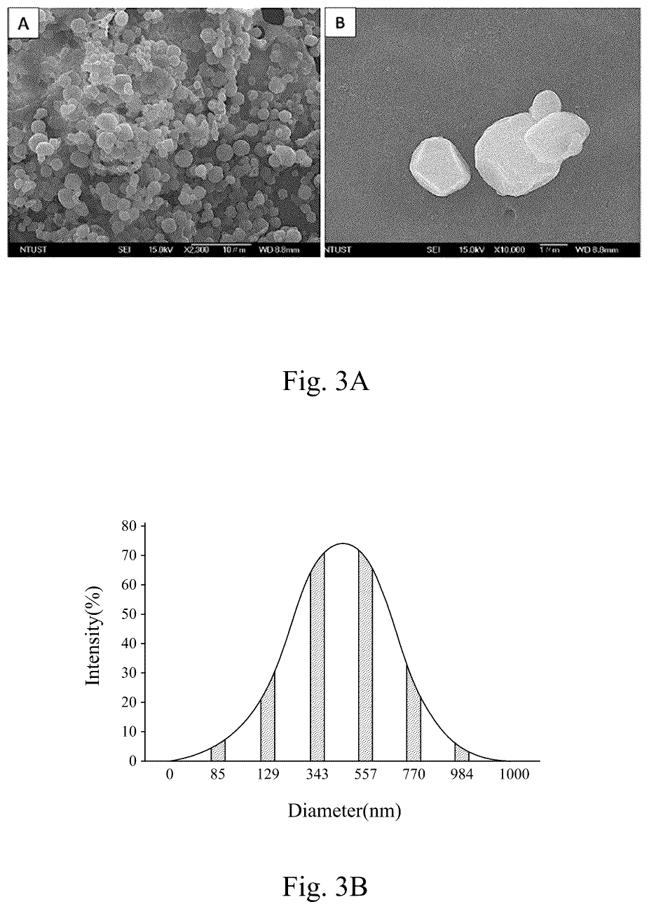

As shown in FIGS. 3A and 3B, a diameter of each of the extracellular interstitial nanoparticles is less than 5000 nanometers (nm) (i.e., 5 μm). In some embodiments, the diameter of each of the extracellular matrix nanoparticles is in a range from 400 nm to 2000 nm. In some embodiments, the diameter of each of the extracellular matrix nanoparticles is in the range from 85 nm to 1000 nm. In some embodiments, the diameter of each of the extracellular matrix nanoparticles includes 85 nm, 129 nm, 343 nm, 557 nm, 770 nm, 984 nm, 1000 nm or any value between any two foregoing diameter values. In addition, since each of the extracellular matrix nanoparticles may be substantially circular, elliptical, or irregular in shape, the diameter of the extracellular matrix nanoparticles is referred to a maximum outer diameter of each of the extracellular matrix nanoparticles.

### II. An Immunomodulatory Effect of the Extracellular Interstitial Nanoparticles Through In Vitro Studies was Detected.

The extracellular matrix (ECM) is known to be configured to support cell attachment, proliferation, and differentiation. Therefore, if the extracellular matrix nanoparticles may be used as a scaffold to anchor stem cells, the extracellular matrix nanoparticles may stay in lungs for a longer period of time, so that the immunomodulatory effect may exert on immune cells. In this way, the extracellular matrix nanoparticles may be configured for a method of preventing or treating a lung disease.

Through a cell migration assay (Transwell assay), Experiment group 1 and Control group 1 are as follows. In Control group 1, macrophages (such as 5×10⁴ RAW 264.7 cells) and culture medium biological buffer (4-(2-hydroxyethyl)-1-piperazineethanesulfonic acid (HEPES) buffer culture medium) were placed in an upper chamber of a culture dish, and a diameter of aperture of the upper chamber was 4 μm. Stem cells (such as 2×10⁵ dental pulp stem cells (DPSC)) and culture medium (including 50% (v/v) standard medium (SM) and 50% (v/v) conditioned medium (CM)) were added into a lower chamber of the culture dish. The difference between Experiment group 1 and Control group 1 was that in Experiment group 1, 200 microliters (μL) of the extracellular matrix nanoparticles were added into a lower chamber of, and then stem cells (such as 2×10⁵ DPSCs) were seeded on surfaces of the extracellular matrix nanoparticles.

Next, after Experiment group 1 and Control group 1 were cultured for 44 hours, Experiment group 1 and Control group 1 were respectively divided into a group stimulated with lipopolysaccharide (LPS) (10 ng/ml) of Escherichia coli for 20 hours, and a group not stimulated with LPS (without LPS).

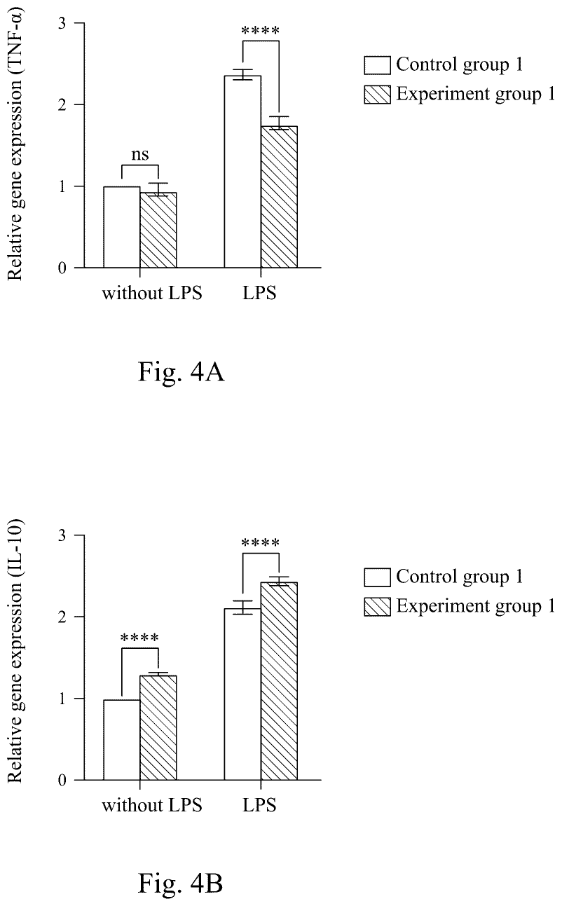

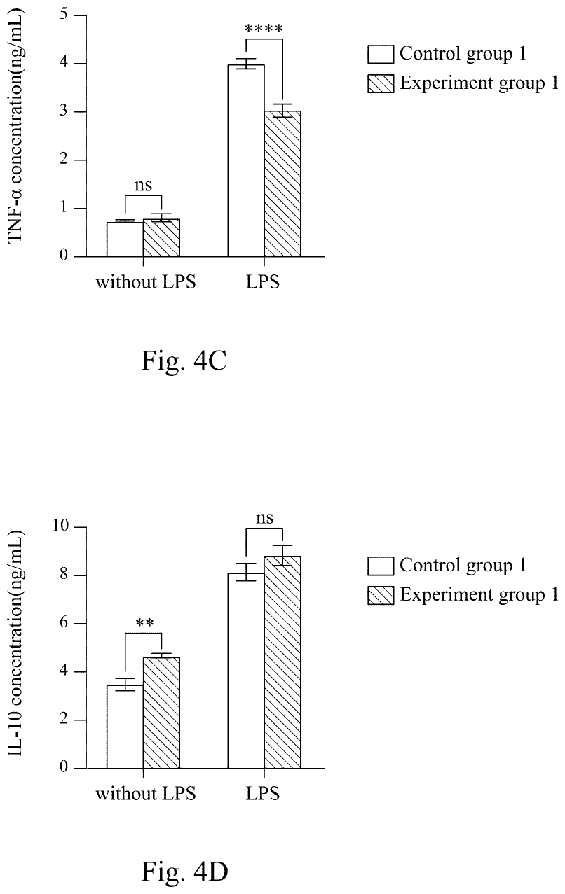

Further, the culture medium in the lower chamber was measured through quantitative real-time PCR (qPCR) and enzyme-linked immunosorbent assay (ELISA) to understand the immunomodulatory effect of the extracellular interstitial nanoparticles on RAW 264.7 cells. Please refer to FIGS. 4A to 4D. FIGS. 4A and 4B are statistical bar charts of IL-10 and TNF-α in Experiment group 1 and Control group 1 respectively obtained through qPCR, and FIGS. 4C and 4D are statistical bar charts of IL-10 and TNF-α in Experiment group 1 and Control group 1 respectively obtained through ELISA. As shown in FIGS. 4A to 4D, it can be seen that the groups not stimulated with LPS have no obvious immunomodulatory effect on macrophages. However, when the cells (Experiment group 1 and Control group 1) are stimulated by LPS, compared with control group 1, the gene expression and protein concentration of TNF-α in Experiment group 1 are significantly reduced, while the gene expression of IL-10 is significantly increased and the protein concentration of IL-10 also showed an upward trend. That is, under inflammatory conditions, Experiment group 1 significantly reduces the concentration of TNF-α promoting inflammatory cytokines, and Experiment group 1 promotes the release of the anti-inflammatory cytokine, IL-10.

### III. An Efficacy of the Extracellular Matrix Nanoparticles Provided by Embodiments of the Present Disclosure for Preventing or Treating Lung Diseases Through In Vivo Studies is Proven.

In recent years, since no awfully effective drug has been released for respiratory-related diseases (such as COVID 19, H1N1, etc.). Moreover, the research and development of new drugs is quite complex, which makes the research and development process long and expensive. In addition, higher toxic chemicals and small molecule drugs have unpredictable toxicological reactions. Therefore, bionic drugs that imitate the natural regulatory mechanisms of the human body may not only overcome the bottleneck of traditional drugs in treating some disease targets, but also have relatively stable efficacy and safety. Accordingly, the extracellular matrix nanoparticles manufactured through the porcine bladder tissues and the porcine lung tissues in this disclosure are biopharmaceuticals. Therefore, compared with traditional drugs, the extracellular matrix nanoparticles are relatively stable in terms of safety and rejection, and the extracellular matrix nanoparticles are easier to be developed successfully.

Firstly, it should be mentioned that when the size of each of the extracellular matrix nanoparticles is greater than 5 μm, the extracellular matrix nanoparticles may get stuck in an upper respiratory tract. When the size of each of the extracellular matrix nanoparticles is in the range from 1 μm to 5 μm, the extracellular matrix nanoparticles may reach into the deep lungs, and the extracellular matrix nanoparticles are not easily exhaled with the expiratory airflow. Thus, an absorption rate of the drug may be decreased. When the size of each of the extracellular matrix nanoparticles is less than 1 μm, the extracellular matrix nanoparticles may reach the alveoli through the diffusion mechanism and enter the alveolar cells. In this way, it is more conducive to the absorption of extracellular matrix nanoparticles by the alveolar cells. Therefore, the extracellular matrix nanoparticles configured for the usage for the manufacture of a medicament for preventing or treating a lung disease, such as acute lung injury, pulmonary fibrosis or pneumonia, have the diameter of less than 5 μm. In some embodiments, the size of each of the extracellular matrix nanoparticles is in the range from 0.4 μm to 2 μm.

As shown in Table 1 below, 7-week-old rats of Sprague-Dawley (SD) were divided into the following groups, and the rats were administered by an intratracheal administration. Control group C2-1: the rats were provided with 200 μL of PBS (i.e., without stimulation and without treatment). Control group C2-2: the rats were provided with 200 μL of the extracellular matrix nanoparticles with a concentration of 8 mg/mL (i.e., without stimulation but providing the extracellular matrix nanoparticles). Experiment group E2-1: the rats were stimulated with 200 μL of LPS (i.e., providing stimulation but without treatment). Experiment group E2-2: after the rats were stimulated with 200 μL of LPS, the rats were administered 200 μL of PBS. Experiment group E2-3 (as a prevention group): the rats were administered 200 μL of the extracellular matrix nanoparticles with the concentration of 8 mg/mL, and then the rats were stimulated with 200 μL of LPS. Experiment group E2-4: after the rats were stimulated with 200 μL of LPS, the rats were administered 200 μL of the extracellular matrix nanoparticles with the concentration of 8 mg/mL.

TABLE 1

| | | Control | | | | | |
|---|---|---|---|---|---|---|---|
| | | group | | Experiment group | | | |
| | | C2-1 | C2-2 | E2-1 | E2-2 | E2-3 | E2-4 |
| Without | PBS | ◯ | X | | | | |
| stimulation | Extracellular matrix | X | ◯ | | | | |
| | nanoparticle | | | | | | |
| Stimulation | LPS | | | ◯ | ◯ | ◯ | ◯ |
| | | | | | | (Second) | (First) |
| | PBS | | | X | ◯ | X | X |
| | Extracellular matrix | | | X | X | ◯ | ◯ |
| | nanoparticle | | | | | (First) | (Second) |

*Note:
◯is referred to the reagent was provided to the rats; X is referred to the reagent was not provided to the rats.

In some embodiments, the acute lung injury in the rats was prevented or treated through the intratracheal administration or a spray administration.

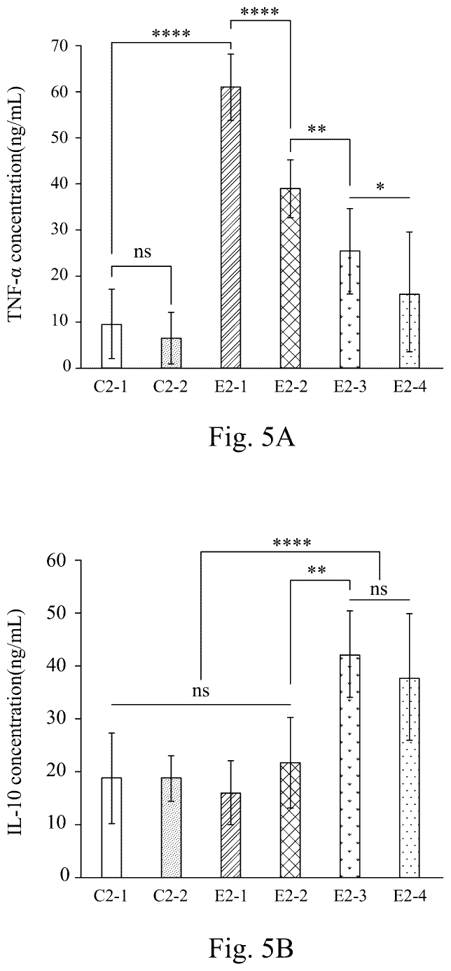

Next, after 6 hours, the rats were sacrificed. Subsequently, the alveoli of the rats were lavaged, and the lavage fluid of each group was analyzed by ELISA to obtain FIGS. 5A and 5B. FIG. 5A is a bar chart related to a concentration of TNF-α in the lavage fluid of each group, and FIG. 5B is a bar chart related to a concentration of IL-10 in the lavage fluid of each group.

As shown in FIGS. 5A and 5B, compared with Control groups C2-1 and C2-2, TNF-α in Experiment group E2-1 is significantly increased. That is, there is an inflammatory response. However, compared with Experiment group E2-1, TNF-α in Experiment group E2-3 and Experiment group E2-4 is significantly inhibited. Furthermore, compared with Experiment group E2-1, it can be known that IL-10 in Experiment group E2-3 and Experiment group E2-4 is significantly increased. Therefore, it can be seen from FIGS. 5A and 5B that the extracellular matrix nanoparticles have anti-inflammatory effects.

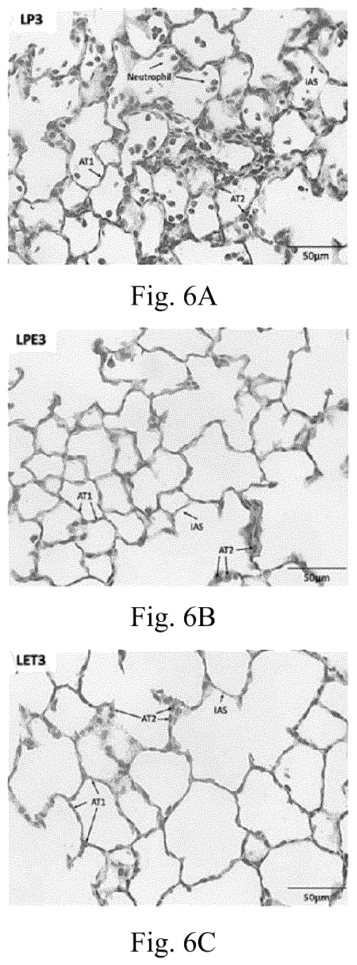

Further, please refer to FIGS. 6A to 6C. FIG. 6A is a section staining figure of an alveolar of Experiment group 2-2, FIG. 6B is a section staining figure of an alveolar of Experiment group 2-3 (i.e., the prevention group), and FIG. 6C is a section staining figure of an alveolar of Experiment group 2-4. As shown in FIGS. 6A to 6C, compared to Experiment group 2-2, Experiment groups 2-3 and 2-4 have fewer or almost no immunocyte (such as neutrophils), and type-I alveolar cells and type-II alveolar cells are relatively abundant. That is, the extracellular matrix nanoparticles have a significant inhibitory effect on neutrophils and may help repair alveolar cells.

Additionally, in some embodiments, a hydrophilic pharmaceutical composition is coated with each of the extracellular matrix nanoparticles. In some embodiments, the hydrophilic pharmaceutical composition includes theophylline, caffeine, aminophylline, dyphylline, or a combination thereof. In some embodiments, a hydrophobic pharmaceutical composition is coated with each of the extracellular matrix nanoparticles. In some embodiments, the hydrophobic pharmaceutical composition includes proteins, DNA, RNA, exosomes, propranolol, camphotosin analogs, silbylin, docetaxel, doxorubicin, naproxen, or a combination thereof.

In addition, since the extracellular matrix nanoparticles are delivered through the biological fluid, such as blood, the extracellular matrix nanoparticles can be applied to a blood-flow-related organ damage or/and disease. Thus, a method of preventing or treating a blood-flow-related organ damage or/and disease is also provided by this disclosure. The method of preventing or treating a blood-flow-related organ damage or/and disease includes administering to a subject in need thereof an effective amount of the plurality of extracellular matrix nanoparticles. Moreover, regardless of a higher rate or a lower rate of blood flow, the extracellular matrix nanoparticles can be delivered through the blood. The higher rate of blood flow is referred to a blood flow to an organ, such as heart, liver, kidney, or brain, is greater than 500 mL per minutes (mL/min), or greater than 15% to 20% in a recumbent man (i.e., at rest). The lower rate of blood flow is referred to a blood flow to an organ, such as bladder, is less than 500 L/min, or less than 15% in a recumbent man (i.e., at rest).

VI. Experiments were Related to the Extracellular Matrix Nanoparticles for Repairing Hair Follicles.

Hair follicles extracted from the dermal papillae of rats (Wistar) were used to conduct a hair follicles growth experiment. A method of repairing hair follicles including the extracellular matrix nanoparticles is proven through Control group 4 and Experiment group 4. In Control group 4, the hair follicles were placed into a 24-well culture plate containing 100 μL of PBS per well (one hair follicle in one well), and 1000 μL of DMEM culture medium was added into each well. Then, DMEM culture medium was replaced once a day, and a growth variation of the hair follicles was observed every ten days. In Experiment group 4, the hair follicles were placed into a 24-well culture plate containing 100 μL of extracellular matrix nanoparticles per well, and 1000 μL of DMEM culture medium was added into each well. A concentration of the extracellular matrix nanoparticles in each well was 2 mg/mL. Then, DMEM culture medium was replaced once a day, and a growth variation of the hair follicles was observed every ten days.

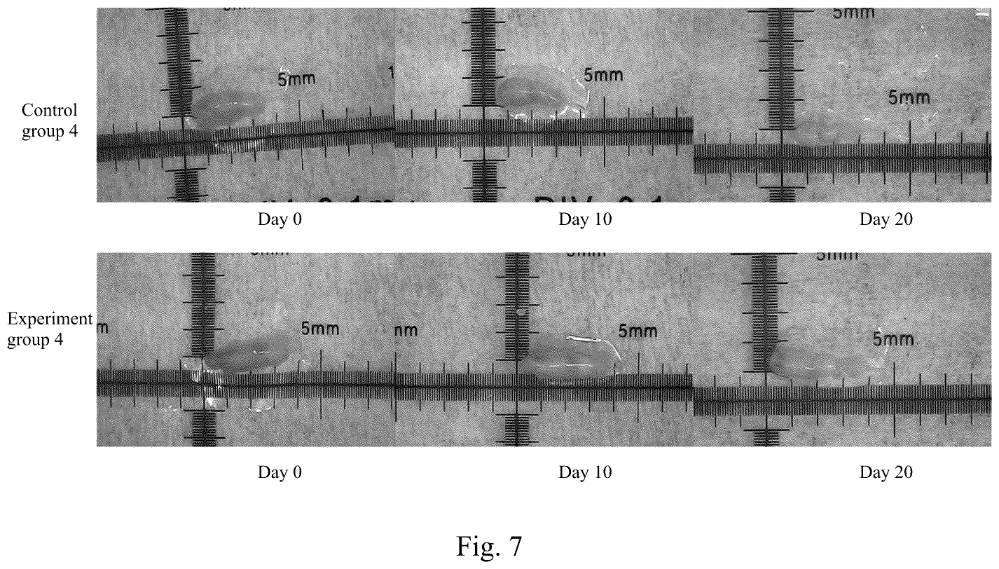

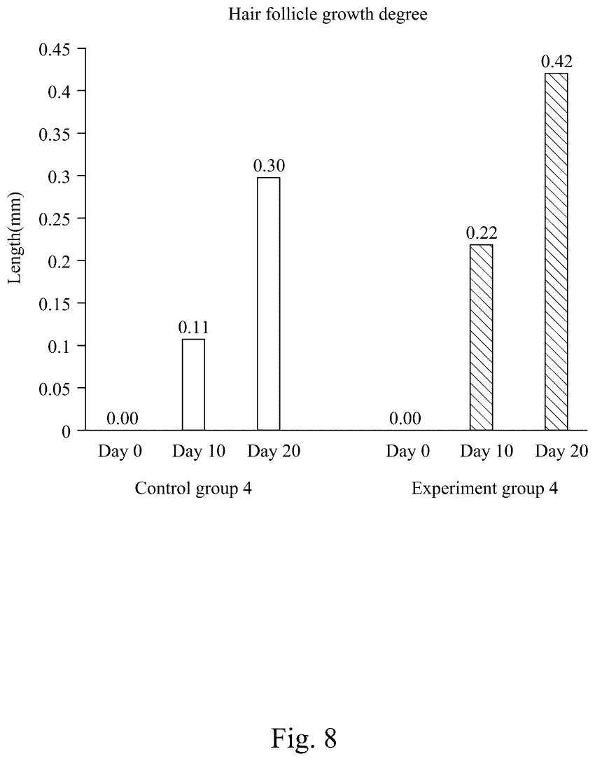

As shown in a diagram of the growth variation of the hair follicles in FIG. 7 and normalized hair follicle growth degrees in FIG. 8, compared with Control group 4, from the 10^{th} day, a hair follicle growth degree of Experiment group 4 was twice as much as a hair follicle growth degree of Control group 4. Moreover, on the 20^{th} day, the hair follicles of Experiment group 4 still continued to grow. Therefore, as shown in FIGS. 7 and 8, it can see that the extracellular matrix nanoparticles repairs hair follicles.

Although the present disclosure has been described with reference to the embodiments above, it is not used to limit this disclosure. It will be apparent to those skilled in the art that various modifications and variations can be made to the structure of the present disclosure without departing from the scope or spirit of the disclosure. Therefore, the scope of protection of this disclosure shall be subject to the scope of the appended claims.

What is claimed is:

1. A method for preparing extracellular matrix nanoparticles, comprising:
providing visceral tissues of a pig;
homogenizing the visceral tissues;
decellularizing the visceral tissues to obtain an extracellular matrix;
lyophilizing the extracellular matrix, and grinding the extracellular matrix lyophilized into powder;
performing a digestion process on the extracellular matrix ground through an acidic solution to produce an acidic extracellular matrix;
performing a neutralization process on the acidic extracellular matrix through an alkaline solution to produce an extracellular matrix hydrogel;
culturing the extracellular matrix hydrogel to gelatinize the extracellular matrix hydrogel; and
performing a sonication process on the extracellular matrix hydrogel to make the extracellular matrix hydrogel into a plurality of extracellular matrix nanoparticles.

2. The method of claim 1, wherein the visceral tissues are bladder tissues or lung tissues.

3. The method of claim 1, wherein a diameter of each of the plurality of extracellular matrix nanoparticles is less than 5 micrometers.

4. The method of claim 1, wherein after the decellularizing the visceral tissues, more than 98% of dsDNA is removed from the visceral tissues.

5. The method of claim 1, wherein a pH value of the acidic solution is in a range from 3 to 4.

6. The method of claim 1, wherein after the neutralization process, a pH value of the extracellular matrix hydrogel is in a range from 7.0 to 7.4.

7. The method of claim 1, when a concentration of the extracellular matrix hydrogel is 4 mg/mL, a storage modulus of the extracellular matrix hydrogel is in a range from 25 Pa to 35 Pa.

8. The method of claim 1, when a concentration of the extracellular matrix hydrogel is in a range from 6 mg/mL to 8 mg/mL, a storage modulus of the extracellular matrix hydrogel is in a range from 110 Pa to 120 Pa.

9. The method of claim 1, wherein the sonication process is performed through an ultrasonic homogenizer.

10. A method of preventing or treating a lung disease, comprising administering to a subject in need thereof an effective amount of a plurality of extracellular matrix nanoparticles, wherein a diameter of each of the extracellular matrix nanoparticles is less than 5 micrometers.

11. The method of claim 10, wherein the diameter of each of the extracellular matrix nanoparticles is in a range from 0.4 μm to 2 μm.

12. The method of claim 10, wherein a hydrophilic pharmaceutical composition is coated with each of the extracellular matrix nanoparticles.

13. The method of claim 10, wherein a hydrophobic pharmaceutical composition is coated with each of the extracellular matrix nanoparticles.

14. The method of claim 10, wherein the lung disease comprises acute lung injury, pulmonary fibrosis and pneumonia.

15. The method of claim 10, wherein an administration way of the extracellular matrix nanoparticles comprises an intratracheal administration and a spray administration.

16. The method of claim 10, wherein the effective amount of the plurality of extracellular matrix nanoparticles increases an anti-inflammatory effect through promotion of IL-10 release and inhibition of TNF-α release.

17. The method of claim 10, wherein the effective amount of the plurality of extracellular matrix nanoparticles decreases neutrophils.

18. A method of repairing hair follicles, comprising administering to a subject in need thereof an effective amount of a plurality of extracellular matrix nanoparticles.
